(19) United States
(12) Patent Application Publication
(10) Pub. No.: US 2019/0337870 A1
(43) Pub. Date: Nov. 7, 2019

(54) METHOD OF STARTING UP A REACTOR FOR THE OXIDATIVE DEHYDROGENATION OF N-BUTENES

(71) Applicants: BASF SE, Ludwigshafen am Rhein (DE); LINDE AG, München (DE)

(72) Inventors: JAN UNGELENK, Ludwigshafen am Rhein (DE); Oliver HAMMEN, Ludwigshafen am Rhein (DE); Ulrich HAMMON, Ludwigshafen am Rhein (DE); Rainer ECKRICH, Ludwigshafen am Rhein (DE); Signe UNVERRICHT, Ludwigshafen am Rhein (DE); Christian WALSDORFF, Ludwigshafen am Rhein (DE); Heinz BOELT, München (DE); Hendrik REYNEKE, München (DE); Christine TOEGEL, München (DE); Anton WELLENHOFER, München (DE); Ulrike WENNING, München (DE)

(21) Appl. No.: 16/324,315

(22) PCT Filed: Aug. 8, 2017
(86) PCT No.: PCT/EP2017/070113

§ 371 Date: Feb. 8, 2019

(30) Foreign Application Priority Data
Aug. 9, 2016 (EP) 16183316.5

(51) Int. Cl. C07C 5/48; C07C 7/11; C07C 11/167

(52) U.S. Cl. CPC C07C 5/48; C07C 11/167; C07C 7/11

(57) ABSTRACT

Process for preparing butadiene from n-butenes, which has a start-up phase and an operating phase and the operating phase of the process comprises the steps:
A) provision of a feed gas stream a1 comprising n-butenes;
B) introduction of the feed gas stream a1 comprising n-butenes, an oxygen-comprising gas stream a2 and an oxygen-comprising recycle gas stream d2 into at least one oxidative dehydrogenation zone and oxidative dehydrogenation of n-butenes to butadiene, giving a product gas stream b comprising butadiene;
C) cooling and compression of the product gas stream b, giving at least one aqueous condensate stream c1 and a gas stream c2 comprising butadiene;
D) introduction of the gas stream c2 into an absorption zone and separation of incondensable and low-boiling gas constituents as gas stream d from the gas stream c2 by absorption of the C₄ hydrocarbons in an absorption medium, giving an absorption medium stream loaded with C₄ hydrocarbons and the gas stream d, and recirculation of the gas stream d as recycle gas stream d2 to the oxidative dehydrogenation zone,
where the start-up phase comprises the steps, in the order i) to iv):
i) introduction of a gas stream d2′ having a composition corresponding to the recycle gas stream d2 in the operating phase into the dehydrogenation zone and setting of the recycle gas stream d2 to at least 70% of the total volume flow in the operating phase;
ii) optionally additional introduction of a steam stream a3 into the dehydrogenation zone;
iii) additional introduction of the feed gas stream a1 comprising butenes at a lower volume flow than in the operating phase and raising of this volume flow until at least 50% of the volume flow of the feed gas stream a1 in the operating phase has been attained, with the total gas flow through the dehydrogenation zone corresponding to not more than 120% of the total gas flow during the operating phase;
iv) additional introduction, when at least 50% of the volume flow of the feed gas stream a1 comprising butenes in the operating phase has been attained, of an oxygen-comprising stream a2 at a lower volume flow than in the operating phase and raising of the volume flows of the feed gas streams a1 and a2 until the volume flows in the operating phase have been attained, with the total gas flow through the dehydrogenation zone corresponding to not more than 120% of the total gas flow during the operating phase.

The invention relates to a method of starting up a reactor for preparing 1,3-butadiene from n-butenes by oxidative dehydrogenation (ODH).

Butadiene is an important basic chemical and is used, for example, for producing synthetic rubbers (butadiene homopolymers, styrene-butadiene rubber or nitrile rubber) or for producing thermoplastic terpolymers (acrylonitrile-butadiene styrene copolymers). Butadiene is also converted into sulfolane, chloroprene and 1,4-hexamethylenediamine (via 1,4-dichlorobutene and adiponitrile). Furthermore, dimerization of butadiene can give vinylcyclohexene which can be dehydrogenated to styrene.

Butadiene can be prepared by thermal dissociation (steam cracking) of saturated hydrocarbons, with naphtha usually being employed as raw material. Steam cracking of naphtha gives a hydrocarbon mixture composed of methane, ethane, ethene, acetylene, propane, propene, propyne, allene, butanes, butenes, butadiene, butynes, methylallene, C₅-hydrocarbons and higher hydrocarbons.

Butadiene can also be obtained by oxidative dehydrogenation of n-butenes (1-butene and/or 2-butene). Any mixture comprising n-butenes can be used as feed gas for the oxidative dehydrogenation (oxydehydrogenation, ODH) of n-butenes to form butadiene. For example, it is possible to use a fraction which comprises n-butenes (1-butene and/or 2-butene) as main constituent and has been obtained from the C₄ fraction from a naphtha cracker by removal of butadiene and isobutene. Furthermore, gas mixtures which comprise 1-butene, cis-2-butene, trans-2-butene or mixtures thereof and have been obtained by dimerization of ethylene can also be used as feed gas. In addition, gas mixtures which comprise n-butenes and have been obtained by catalytic fluidized-bed cracking (Fluid Catalytic Cracking, FCC) can be used as feed gas.

The reaction of the gas streams comprising butenes is generally carried out in industry in shell-and-tube reactors which are operated in a salt bath as heat transfer medium. The product gas stream is cooled downstream of the reactor by direct contact with a coolant in a quenching stage and is subsequently compressed. The C₄ components are then absorbed in an organic solvent in an absorption column. Inert gases, low boilers, CO, CO₂ and others leave the column at the top. This overhead stream is partly fed as recycle gas to the ODH reactor. Hydrocarbons and oxygen can produce an explosive atmosphere. The concentration of the combustible gas constituents (mainly hydrocarbons and CO) can be below the lower explosion limit (LEL) or above the upper explosion limit (UEL) in order to avoid ignitable mixtures. Below the lower explosion limit, the oxygen concentration can be selected freely without an explosive gas mixture being able to form. However, the concentration of feed gas is then low, which is economically disadvantageous. For this reason, a reaction using a reaction gas mixture above the upper explosion limit is preferred. Here, whether an explosion can occur depends on the oxygen concentration. Below a particular oxygen concentration, the LOC (Limiting Oxygen Concentration), the concentration of combustible gas constituents can be selected freely without an explosive gas mixture being able to form. Both LEL, UEL and LOC are temperature- and pressure-dependent.

On the other hand, precursors of carbonaceous material can, depending on the oxygen concentration, be formed in the oxidative dehydrogenation of n-butenes to butadiene and these can ultimately lead to carbonization, deactivation and irreversible destruction of the multimetal oxide catalyst. This is still possible when the oxygen concentration in the reaction gas mixture of the oxydehydrogenation is above the LOC at the inlet into the reactor.

The necessity of an excess of oxygen for such catalyst systems is generally known and is reflected in the process conditions when using such catalysts. As examples, the relatively recent work by Jung at al. (Catal. Surv. Asia 2009, 13, 78-93; DOI 10.1007/s10563-009-9069-5 and Applied Catalysis A: General 2007, 317, 244-249; DOI 10.1016/j.apcata.2006.10.021) may be mentioned.

However, the presence of high oxygen concentrations in addition to hydrocarbons such as butane, butene and butadiene or the organic absorption media used in the work-up section is associated with risks. Thus, explosive gas mixtures can be formed. If the process is operated close to the explosive range, it is not always technically possible to prevent this range being entered due to fluctuations in the process parameters. The period of time in which the reactor is started up and reaction gas mixture is passed through it is particularly critical in respect of risk of explosion and carbonization of the catalyst.

Processes for the oxidative dehydrogenation of butenes to butadiene are known in principle.

US 2012/0130137 A1, for example, describes a process of this type using catalysts which comprise oxides of molybdenum, bismuth and generally further metals. A critical minimum oxygen partial pressure in the gas atmosphere is necessary for the long-term activity of such catalysts for the oxidative dehydrogenation in order to avoid excessive reduction and thus a decrease in performance of the catalysts. For this reason, it is generally also not possible to employ a stoichiometric input of oxygen or a complete oxygen conversion in the oxydehydrogenation reactor (ODH reactor). For example, an oxygen content of from 2.5 to 8% by volume in the product gas is described in US 2012/0130137 A1.

In particular, the problems of formation of any explosive mixtures after the reaction step are discussed in paragraph [0017]. It may in particular be pointed out that in the case of a “rich” mode of operation above the upper explosion limit in the reaction section, there is the problem that after absorption of a major part of the organic constituents in the work-up the gas composition crosses over into the explosive range when there is a transition from a rich gas mixture to a lean gas mixture. Thus, it is stated in paragraphs 0061-0062 that it is necessary according to the invention for the concentration of combustible gas constituents in the gas mixture introduced into the oxidative dehydrogenation reactor to be above the upper explosion limit and, during starting-up of the oxidative dehydrogenation reaction, the oxygen concentration in the mixed gas at the reactor inlet is firstly set to a value below the limiting oxygen concentration (LOC) by firstly setting the amount of oxygen-comprising gas and steam introduced into the reactor and then commencing the introduction of combustible gas (essentially feed gas). The amount of oxygen-comprising gas introduced, for example air, and combustible gas can subsequently be increased so that the concentration of combustible gas constituents in the mixed gas is greater than the upper explosion limit. As soon as the amount of combustible gas constituents and oxygen-comprising gas introduced increases, the amount of nitrogen and/or steam introduced is reduced so that the amount of mixed gas introduced remains stable.

It may also be pointed out that there is a risk of catalyst deactivation by carbonization in the case of continuous lean operation in the reaction section. However, US 2012/0130137 A1 does not indicate a solution to this problem.

In paragraph [0106], it is indicated incidentally how the occurrence of explosive atmospheres in the absorption step can, for example, be avoided by dilution of the gas stream with nitrogen before the absorption step. Nothing further is said about the problem of the formation of explosive gas mixtures in the more detailed description of the absorption step in paragraphs [0132] ff.

The conditions which have to be adhered to in order to prevent carbonization of the catalyst are not described in the document. Furthermore, the document does not relate to a process carried out in the gas recycle mode. Furthermore, the streams are adjusted in succession, which means complicated operation.

JP 2016-69352 likewise discusses a process for the oxidative dehydrogenation of butenes to butadiene and describes the problem that there is a risk of catalyst deactivation by carbonization during low-oxygen operation in the reaction section. At the same time, mention is made of the problem that the concentration of combustion gas and oxygen cannot be chosen at will in view of the formation of possible explosive mixtures. In particular, it is stated in paragraphs [0045-0046] that the LOC increases with increasing proportions of carbon dioxide when a mixture of nitrogen and carbon dioxide is selected as inert gas. However, this procedure is economically disadvantageous since the provision of inert gases is expensive and carbon dioxide in addition to nitrogen represents a further inert gas which has to be provided.

JP 2010-280653 describes the start-up of an ODH reactor. The reactor should be started up without catalyst deactivation or an increase in the pressure drop occurring. This is said to be made possible by running up the reactor to more than 80% of full load within 100 hours. It is stated in paragraph 0026 that, according to the invention, the amount of raw material gas supplied to the reactor per unit time is set to more than 80% of the maximum permissible amount to be supplied during start-up of the reaction less than 100 hours after supply of raw material gas to the reactor is commenced, and during this time the amount of the nitrogen gas, of the gas comprising elemental oxygen and of steam introduced together with the raw material gas into the reactor is regulated in such a way that the composition of the mixed gas composed of raw material gas, nitrogen gas, gas comprising elemental oxygen and steam does not go into the explosive range. The document does not describe the conditions which have to be adhered to in order to prevent carbonization of the catalyst. Furthermore, the document does not relate to a process operated in the gas recycle mode. Furthermore, the document does not consider the explosion problems in the work-up section of the process.

EP 1 180 508 describes the start-up of a reactor for catalytic gas-phase oxidation. The oxidation of propylene to acrolein is specifically described. A process in which a range in which the oxygen content of the reaction gas mixture is greater than the LOC and the concentration of combustible gas constituents is less than the LEL is passed through during start-up of the reactor is described. In steady-state operation, the O₂ concentration is then less than the LOC and the concentration of combustible gas constituents is greater than the UEL.

DE 1 0232 482 describes a method of safely operating an oxidation reactor for the gas-phase partial oxidation of propylene to acrolein and/or acrylic acid using a computer-aided shutdown mechanism. This is based on the recording of an explosion graph and determination of the concentration of C₄ and O₂ by measuring of the O₂ and C-hydrocarbon concentration in the recycle gas and the volume flows of recycle gas, C₃-hydrocarbon stream and oxygen-comprising gas. The start-up of the reactor is described in paragraphs 0076-0079. It is said in paragraph 0079 that opening of the introduction of firstly air and then propene is permitted only when the inflowing amount of diluent gas (steam and/or recycle gas) has risen to a minimum value which is, for example, 70% of the maximum possible amount of air which can be fed in. The concentration of O₂ in the recycle gas is already identical to steady-state operation (3.3% by volume) during the start-up procedure.

WO 2015/104397 discloses a process for preparing butadiene from n-butenes, which has a start-up phase and an operating phase and the operating phase of the process comprises the steps:

A) provision of a feed gas stream a1 comprising n-butenes;

B) introduction of the feed gas stream a1 comprising n-butenes, an oxygen-comprising gas stream a2 and an oxygen-comprising recycle gas stream d2 into at least one oxidative dehydrogenation zone and oxidative dehydrogenation of n-butenes to butadiene, giving a product gas stream b comprising butadiene, unreacted n-butenes, steam, oxygen, low-boiling hydrocarbons, high-boiling secondary components, possibly carbon oxides and possibly inert gases;

C) cooling and compression of the product gas stream b and condensation of at least part of the high-boiling secondary components, giving at least one aqueous condensate stream c1 and a gas stream c2 comprising butadiene, n-butenes, steam, oxygen, low-boiling hydrocarbons, possibly carbon oxides and possibly inert gases;

D) introduction of the gas stream c2 into an absorption zone and separation of incondensable and low-boiling gas constituents comprising oxygen, low-boiling hydrocarbons, possibly carbon oxides and possibly inert gases as gas stream d from the gas stream c2 by substantial absorption of the C₄ hydrocarbons comprising butadiene and n-butenes in an absorption medium, giving an absorption medium stream loaded with C₄ hydrocarbons and the gas stream d, and recirculation, optionally after a purge gas stream p has been separated off, of the gas stream d as recycle gas stream d2 to the oxidative dehydrogenation zone;

where the start-up phase comprises the steps, in the order i) to iv):

i) introduction of an oxygen-comprising gas stream and an inert gas stream into the dehydrogenation zone in such a ratio that the oxygen content of the recycle gas stream d2 corresponds to from 30 to 80% of the oxygen content of the recycle gas stream d2 in the operating phase;

ii) setting of the recycle gas stream d2 to at least 70% of the volume flow of the recycle gas d2 in the operating phase;

iii) optionally introduction, at an initial oxygen content of the recycle gas stream d2 of from 30 to 80% of the oxygen content of the recycle gas stream d2 in the operating phase, of a steam stream a3 into the dehydrogenation zone;

iv) introduction, at an initial oxygen content of the recycle gas stream d2 of from to 80% of the oxygen content of the recycle gas stream d2 in the operating phase, of an oxygen-comprising gas stream a2′ and a feed gas stream a1′ which comprises butenes at lower volume flows than in the operating phase in a ratio k=a2′/a1′ and raising of the volume flows of the gas streams a1′ and a2′ until the volume flows of the gas streams a1 and a2 in the operating phase are attained, with the recycle gas stream d2 being at least 70% and not more than 120% of the volume flow in the operating phase.

Thus, according to WO 2015/104397, the high oxygen content of the recycle gas is, during the start-up procedure, firstly diluted with an inert gas, e.g. nitrogen, to such an extent that the oxygen content of the recycle gas preferably corresponds to from 50 to 60% of the oxygen content in the operating phase. Proceeding from this point, the oxygen content and the content of butenes are increased in such a way that a sufficient distance from the explosion limit is always ensured. The oxygen content is increased by adding an oxygen-comprising gas, preferably air. However, this procedure is economically disadvantageous since the oxygen content of the recycle gas available is firstly decreased by dilution with an additional inert gas and is subsequently increased again by addition of an additional oxygen-comprising gas. Here, it should be noted that the provision of inert gas is expensive and the addition of oxygen-comprising gas, e.g. air, also incurs costs since the air firstly has to be compressed to the required process pressure.

It is an object of the invention to provide a safe and economical method of starting up a reactor for the oxidative dehydrogenation of n-butenes to butadiene and also starting up downstream units for the work-up of the product gas mixture.

The object is achieved by a process for preparing butadiene from n-butenes, which has a start-up phase and an operating phase and the operating phase of the process comprises the steps:

A) provision of a feed gas stream a1 comprising n-butenes;

B) introduction of the feed gas stream a1 comprising n-butenes, an oxygen-comprising gas stream a2 and an oxygen-comprising recycle gas stream d2 into at least one oxidative dehydrogenation zone and oxidative dehydrogenation of n-butenes to butadiene, giving a product gas stream b comprising butadiene, unreacted n-butenes, steam, oxygen, low-boiling hydrocarbons, high-boiling secondary components, possibly carbon oxides and possibly inert gases;

C) cooling and compression of the product gas stream b and condensation of at least part of the high-boiling secondary components, giving at least one aqueous condensate stream c1 and a gas stream c2 comprising butadiene, n-butenes, steam, oxygen, low-boiling hydrocarbons, possibly carbon oxides and possibly inert gases;

D) introduction of the gas stream c2 into an absorption zone and separation of incondensable and low-boiling gas constituents comprising oxygen, low-boiling hydrocarbons, possibly carbon oxides and possibly inert gases as gas stream d from the gas stream c2 by substantial absorption of the C₄ hydrocarbons comprising butadiene and n-butenes in an absorption medium, giving an absorption medium stream loaded with C₄ hydrocarbons and the gas stream d, and recirculation, optionally after a purge gas stream p has been separated off, of the gas stream d as recycle gas stream d2 to the oxidative dehydrogenation zone;

where the start-up phase comprises the steps, in the order i) to iv):

i) introduction of a gas stream d2′ having a composition corresponding to the recycle gas stream d2 in the operating phase into the dehydrogenation zone and setting of the recycle gas stream d2 to at least 70% of the total volume flow in the operating phase;

ii) optionally additional introduction of a steam stream a3 into the dehydrogenation zone;

iii) additional introduction of the feed gas stream a1 comprising butenes at a lower volume flow than in the operating phase and raising of this volume flow until at least 50% of the volume flow of the feed gas stream a1 in the operating phase has been attained, with the total gas flow through the dehydrogenation zone corresponding to not more than 120% of the total gas flow during the operating phase;

iv) additional introduction, when at least 50% of the volume flow of the feed gas stream a1 comprising butenes in the operating phase has been attained, of an oxygen-comprising stream a2 at a lower volume flow than in the operating phase and raising of the volume flows of the feed gas streams a1 and a2 until the volume flows in the operating phase have been attained, with the total gas flow through the dehydrogenation zone corresponding to not more than 120% of the total gas flow during the operating phase.

The start-up procedure according to the invention has the advantage over the mode of operation described in WO 2015/104397 that oxygen-rich conditions prevail even at the beginning of the start-up phase since the recycle gas is not diluted with an inert gas. This counters carbonization of the catalyst. The conditions in respect of load, gas velocity, residence time and composition of the recycle gas stream during the start-up phase correspond more to the conditions during the operating phase. Since the gas velocity is essentially constant, a hot spot which forms does not migrate within the reactor. Steady-state operation and optimal performance of the catalyst in respect of the space-time yield and selectivity are reached more quickly overall.

In step i), the recycle gas stream d2 is preferably set to from 90 to 110% of the total volume flow in the operating phase. The total volume flow is the sum of the volume flows of the streams a1, a2, d2 and optionally a3. In a particularly preferred embodiment, the recycle gas stream d2 is set to 95-105% of the total volume flow in the operating phase; the recycle gas stream d2 is particularly preferably set to 100% of the total volume flow in the operating phase. The recycle gas stream d2 set is reduced in the subsequent steps iii) and iv) in such a way that the total gas flow through the dehydrogenation zone, i.e. the sum of the streams a1, a2, d2 and optionally a3, during the further start-up phase is at least 70% and not more than 120%, preferably at least 90% and not more than 115%, of the total gas flow during the operating phase. The total gas flow preferably remains substantially constant during the start-up phase and varies by not more than +/−10% by volume, in particular +/−5% by volume, i.e. during the start-up phase is preferably from 90 to 110% by volume, in particular from 95 to 105% by volume, of the total gas flow during the operating phase.

In step i), a gas stream d2′ having a composition corresponding to the recycle gas stream d2 in the operating phase is fed into the dehydrogenation zone. As gas stream d2′, part of the recycle gas stream is preferably taken off from one or more reactors operated in parallel for preparing butadiene from n-butenes which are in the operating phase. In this case, the dehydrogenation zone in step B) thus comprises a plurality of reactors, with at least one of the reactors, preferably at least two of the reactors, being in the operating phase and generating a total recycle gas stream from which the gas stream d2′ or d2 is taken off. In general, the gas stream d2′ and accordingly the recycle gas stream d2 comprise from 5.5 to 8.5% by volume of O₂ and from 89.4 to 94.5% by weight of inert gases selected from among nitrogen, noble gases (in particular argon) and carbon oxides (CO, CO₂) in the operating phase. In addition, the gas stream d2′ and accordingly the recycle gas stream d2 can comprise from 0 to 0.5% by volume of steam. Furthermore, the recycle gas stream d2 and accordingly the gas stream d2′ can further comprise from 0 to 1.5% by volume of oxygen-comprising compounds such as acrolein and from 0 to 0.1% by weight of hydrocarbons.

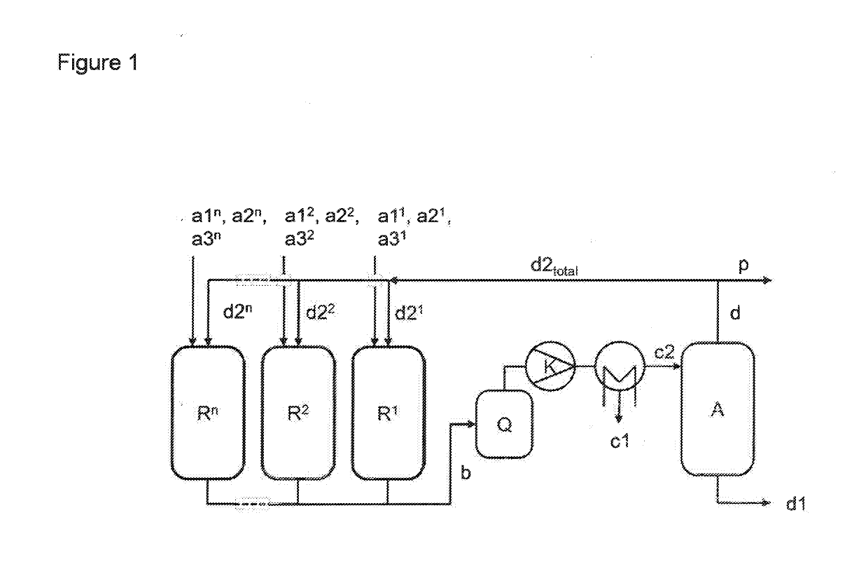

One variant having a plurality of reactors is depicted in FIG. 1. Here,

R¹, R², Rⁿ are n reactors which are operated in parallel and of which R¹ is in the start-up phase and R², Rⁿ are in the operating phase,

a1¹, a2¹, a3¹, a1², a2², a3², a1ⁿ, a2ⁿ, a3ⁿ are the streams a1, a2, a3 assigned to the individual reactors,

d2ₜₒₜₐₗ is the total recycle gas stream,

d2¹, d2², d2ⁿ are the partial recycle gas streams assigned to the individual reactors,

b is the butadiene-comprising C4 product gas stream,

Q is a quenching stage,

K is a compression stage,

c1 is an aqueous condensate stream,

c2 is the butadiene-comprising C4 product gas stream,

A is the absorption stage,

d1 is the butadiene-comprising C4 product gas stream,

d is the total recycle gas stream before a purge stream has been separated off, and

p is the purge stream.

The gas stream d2′ has a composition corresponding to the recycle gas stream d2 in the operating phase when its oxygen content deviates by not more than +/−2% by volume from the oxygen content of the recycle gas stream d2 in steady-state operation.

In step ii), a steam stream a3 can be additionally fed into the dehydrogenation zone. In general, the amount of steam in the dehydrogenation zone during steps ii) to iv) is from 0.5 to 10% by volume, preferably from 1 to 7% by volume. This can also be atmospheric humidity.

In step iii), the feed gas stream a1 comprising butenes is additionally fed into the dehydrogenation zone until at least 50% of the volume flow in the operating phase has been attained. The volume flow is generally increased in steps, for example, commencing at 10% of the volume flow in the operating state, in steps of 10% until at least 50% of the volume flow in the operating state has been attained. The volume flow can also be increased in the form of a ramp. Here, the recycle gas stream d2 is optionally decreased to such an extent that the total gas flow through the dehydrogenation zone corresponds to not more than 120% of the total gas flow during the operating phase.

The content of C₄-hydrocabons (butenes and butanes) in the total gas stream through the dehydrogenation zone is generally from 7 to 9% by volume at the end of step iii).

The volume flow of the feed gas stream a1 comprising butenes can also be increased in step iii) until at least 60% of the volume flow in the operating phase has been attained, but at most until not more than 75% of the volume flow in the operating phase has been attained.

In step iv), an oxygen-comprising stream a2 is, when at least 50% and not more than 75% of the volume flow of the feed gas stream a1 comprising butenes in the operating phase has been attained, fed at a lower volume flow than in the operating phase in addition to the feed gas stream a1 comprising butenes into the dehydrogenation zone and the volume flows of the feed gas streams a1 and a2 are increased until the volume flows in the operating phase have been attained. In general, the volume flow of the oxygen-comprising gas stream a2 is increased in a first step in one or more stages until a ratio of oxygen to hydrocarbons which corresponds to the ratio of oxygen to hydrocarbons in the operating phase has been reached and both the volume streams a1 and a2 are subsequently increased in stages until 100% of the volume flow of each of the gas streams a1 and a2 in the operating phase has been attained, with the ratio of oxygen to hydrocarbons remaining substantially constant and corresponding to the ratio of oxygen to hydrocarbons in the operating phase. The volume flow is generally increased in steps, for example commencing with 50% of the volume flow of the gas stream a1 in the operating state, in steps of, for example, 10%, with the stages for the steps for increasing the volume flow of the gas stream a2 being selected so that the ratio of oxygen to hydrocarbons remains substantially constant during the start-up phase until 100% of the volume flows in the operating state has been attained.

The ratio of oxygen to hydrocarbons during the start-up phase corresponds to the ratio of oxygen to hydrocarbons in the operating phase when it deviates from the latter ratio by not more than 10%. The ratio of oxygen to hydrocarbons in the operating phase is generally from 0.65:1 to 1.5:1, preferably from 0.65:1 to 1.3:1, at an n-butenes content of from 50 to 100% by volume in the feed gas stream a1. The ratio can change in the operating phase.

The content of C₄-hydrocarbons (butenes and butanes) in the total gas stream through the dehydrogenation zone is generally from 7 to 9% by volume at the end of step (iv), and the oxygen content is generally from 12 to 13% by volume.

In general, the pressure in the dehydrogenation zone during the start-up phase is from 1 to 5 bar absolute, preferably from 1.05 to 2.5 bar absolute.

In general, the pressure in the absorption zone during the start-up phase is from 2 to 20 bar, preferably from 5 to 15 bar.

In general, the temperature of the heat transfer medium during the start-up phase is in the range from 220 to 490° C. and preferably from 300 to 450° C. and particularly preferably from 330 to 420° C.

In general, the duration of the start-up phase is from 15 to 2000 minutes, preferably from 15 to 500 minutes and particularly preferably from 20 to 120 minutes. The operating phase then begins.

In general, the step C) comprises the steps Ca) and Cb):

Ca) cooling of the product gas stream b in at least one coolant stage, where the cooling is effected in at least one cooling stage by contacting with a coolant, and condensation of at least part of the high-boiling secondary components;

Cb) compression of the remaining product gas stream b in at least one compression stage, giving at least one aqueous condensate stream c1 and a gas stream c2 comprising butadiene, n-butenes, steam, oxygen, low-boiling hydrocarbons, possibly carbon oxides and possibly inert gases.

In general, the step D) comprises the steps Da) and Db):

Da) separation of incondensable and low-boiling gas constituents comprising oxygen, low-boiling hydrocarbons, possibly carbon oxides and possibly inert gases as gas stream d from the gas stream c2 by absorption of the C₄-hydrocarbons comprising butadiene and n-butenes in an absorption medium, giving an absorption medium stream loaded with C₄-hydrocarbons and the gas stream d, and

Db) subsequent desorption of the C₄-hydrocarbons from the loaded absorption medium stream, giving a C₄ product gas stream d1.

The steps E) and F) are preferably carried out subsequently:

E) fractionation of the C₄ product stream d1 by extractive distillation using a solvent which is selective for butadiene to give a stream e1 comprising butadiene and the selective solvent and a stream e2 comprising n-butenes;

F) distillation of the stream f2 comprising butadiene and the selective solvent to give a stream g1 consisting essentially of the selective solvent and a butadiene-comprising stream g2.

In general, the gas stream d obtained in step Da) is recirculated to an extent of at least 10%, preferably at least 30%, as recycle gas stream d2 to step B).

In general, aqueous coolants or organic solvents or mixtures thereof are used in the cooling stage Ca).

Preference is given to using an organic solvent in the cooling stage Ca). Such solvents generally have a very much greater solvent capability for the high-boiling by-products, which can lead to deposits and blockages in the plant parts downstream of the ODH reactor, than water or alkaline aqueous solutions.

Preferred organic solvents used as coolant are aromatic hydrocarbons, for example toluene, o-xylene, m-xylene, p-xylene, diethylbenzenes, triethylbenzenes, diisopropylbenzenes, triisopropylbenzenes and mesitylene or mixtures thereof. Particular preference is given to mesitylene.

The following embodiments are preferred or particularly preferred variants of the process of the invention:

Stage Ca) is carried out in a number of stages in stages Ca1) to Can), preferably in two stages Ca1) and Ca2). Here, particular preference is given to at least part of the solvent which has passed through the second stage Ca2) being fed as coolant to the first stage Ca1).

Stage Cb) generally comprises at least one compression stage Cba) and at least one cooling stage Cbb). The gas which has been compressed in the compression stage Cba) is preferably brought into contact with a coolant in the at least one cooling stage Cbb). The coolant of the cooling stage Cbb) particularly preferably comprises the same organic solvent which is used as coolant in stage Ca). In a particularly preferred variant, at least part of this coolant which has passed through the at least one coolant stage Cbb) is fed as coolant to stage Ca).

Stage Cb) preferably comprises a plurality of compression stages Cba1) to Cban) and cooling stages Cbb1) to Cbbn), for example four compression stages Cba1) to Cba4) and four cooling stages Cbb1) to Cbb4).

Step D) preferably comprises the steps Da1), Da2) and Db):
Da1) absorption of the C₄-hydrocarbons comprising butadiene and n-butenes in a high-boiling absorption medium, giving an absorption medium stream loaded with C₄-hydrocarbons and the gas stream d,
Da2) removal of oxygen from the absorption medium loaded with C₄-hydrocarbons from step Da) by stripping with an incondensable gas stream, and
Db) desorption of the C₄-hydrocarbons from the loaded absorption medium stream, giving a C₄ product gas stream d1 which consists essentially of C₄-hydrocarbons and comprises less than 100 ppm of oxygen.

The high-boiling absorption medium used in step Da) is preferably an aromatic hydrocarbon solvent, particularly preferably the aromatic hydrocarbon solvent used in step Ca), in particular mesitylene. It is also possible to use, for example, diethylbenzenes, triethylbenzenes, diisopropylbenzenes and triisopropylbenzenes or mixtures comprising these substances.

Embodiments of the process of the invention are shown in FIG. 1 and are described in detail below.

As feed gas stream, it is possible to use pure n-butenes (1-butene and/or cis-/trans-2-butene) or else gas mixtures comprising butenes. It is also possible to use a fraction which comprises n-butenes (1-butene and cis-/trans-2-butene) as main constituent and has been obtained from the C₄ fraction from a naphtha cracker by removal of butadiene and isobutene. Furthermore, it is also possible to use gas mixtures which comprise pure 1-butene, cis-2-butene, trans-2-butene or mixtures thereof and have been obtained by dimerization of ethylene as feed gas. Furthermore, gas mixtures which comprise n-butenes and have been obtained by fluid catalytic cracking (FCC) can be used as feed gas.

In one embodiment of the process of the invention, the feed gas comprising n-butenes is obtained by nonoxidative dehydrogenation of n-butane. Coupling of a nonoxidative catalytic dehydrogenation with the oxidative dehydrogenation of the n-butenes formed enables a high yield of butadiene, based on n-butane used, to be obtained. The nonoxidative catalytic dehydrogenation of n-butane gives a gas mixture which comprises secondary constituents in addition to butadiene, 1-butene, 2-butene and unreacted n-butane. Typical secondary constituents are hydrogen, steam, nitrogen, CO and CO₂, methane, ethane, ethene, propane and propane. The composition of the gas mixture leaving the first dehydrogenation zone can vary greatly as a function of the way in which the dehydrogenation is carried out. Thus, when the dehydrogenation is carried out with introduction of oxygen and additional hydrogen, the product gas mixture has a comparatively high content of steam and carbon oxides. In mode of operation without introduction of oxygen, the product gas mixture from the nonoxidative dehydrogenation has a comparatively high content of hydrogen.

In step B), the feed gas stream comprising n-butenes and an oxygen-comprising gas are fed into at least one dehydrogenation zone (one or more ODH reactors R operated in parallel) and the butenes comprised in the gas mixture are oxidatively dehydrogenated to butadiene in the presence of an oxydehydrogenation catalyst.

The gas comprising molecular oxygen generally comprises more than 10% by volume, preferably more than 15% by volume and even more preferably more than 20% by volume, of molecular oxygen. It is preferably air. The upper limit for the content of molecular oxygen is generally 50% by volume or less, preferably 30% by volume or less and even more preferably 25% by volume or less. In addition, any inert gases can be comprised in the gas comprising molecular oxygen. Possible inert gases are nitrogen, argon, neon, helium, CO, CO₂ and water. The amount of inert gases is in the case of nitrogen generally 90% by volume or less, preferably 85% by volume or less and even more preferably 80% by volume or less. In the case of constituents other than nitrogen, it is generally 10% by volume or less, preferably 1% by volume or less.

To carry out the oxidative dehydrogenation with full conversion of n-butenes, preference is given to a gas mixture which has a molar oxygen:n-butenes ratio of at least 0.5. Preference is given to employing an oxygen:n-butenes ratio of from 1.25 to 1.6. To set this value, the feed gas stream can be mixed with oxygen or at least one oxygen-comprising gas, for example air, and optionally additional inert gas or steam. The oxygen-comprising gas mixture obtained is then fed to the oxydehydrogenation.

Furthermore, inert gases such as nitrogen and also water (as steam) can also be comprised in the reaction gas mixture. Nitrogen can serve to set the oxygen concentration and to prevent formation of an explosive gas mixture, and the same applies to steam. Steam also serves to control carbonization of the catalyst and to remove the heat of reaction.

Catalysts suitable for the oxydehydrogenation are generally based on an Mo—Bi—O-comprising multimetal oxide system which generally additionally comprises iron. In general, the catalyst comprises further additional components such as potassium, cesium, magnesium, zirconium, chromium, nickel, cobalt, cadmium, tin, lead, germanium, lanthanum, manganese, tungsten, phosphorus, cerium, aluminum or silicon. Iron-comprising ferrites have also been proposed as catalysts.

In a preferred embodiment, the multimetal oxide comprises cobalt and/or nickel. In a further preferred embodiment, the multimetal oxide comprises chromium. In a further preferred embodiment, the multimetal oxide comprises manganese.

Examples of Mo—Bi—Fe—O-comprising multimetal oxides are Mo—Bi—Fe—Cr—O— or Mo—Bi—Fe—Zr—O-comprising multimetal oxides. Preferred catalysts are described, for example, in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}P_{0.5}K_{0.1}Oₓ+SiO₂), DE-A 25 30 959 (Mo₁₂BiFe₃Co_{4.5}Ni_{2.5}Cr_{0.5}K_{0.1}Oₓ, Mo_{13.75}BiFe₃Co_{4.5}Ni_{2.5}Ge_{0.5}K_{0.8}Oₓ, Mo₁₂BiFe₃Co_{4.5}Ni_{2.5}Mn_{0.5}K_{0.1}Oₓ and Mo₁₂BiFe₃Co_{4.5}Ni_{2.5}La_{0.5}K_{0.1}Oₓ), U.S. Pat. No. 3,911,039 (Mo₁₂BiFe₃Co_{4.5}Ni_{2.5}Sn_{0.5}K_{0.1}Oₓ), DE-A 25 30 959 and DE-A 24 47 825 (Mo₁₂BiFe₃Co_{4.5}Ni_{2.5}W_{0.5}K_{0.1}Oₓ).

Suitable multimetal oxides and their production are also described in U.S. Pat. No. 4,423,281 (Mo₁₂BiNi₈Pb_{0.5}Cr₃K_{0.2}Oₓ and Mo₁₂Bi_{b}Ni₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiNi_{0.5}Cr₃P_{0.5}Mg_{7.5}K_{0.1}Oₓ+SiO₂) and DE-A 24 40 329 (Mo₁₂BiCo_{4.5}Ni_{2.5}Cr₃P_{0.5}K_{0.1}Oₓ).

Particularly preferred catalytically active multimetal oxides comprising molybdenum and at least one further metal have the general formula (Ia):

Mo₁₂BiₐFe_{b}Co_{c}Ni_{d}CrₑX¹_{f}X²_{g}O_{y} (Ia),

where
X¹ ═Si, Mn and/or Al,
X²═Li, Na, K, Cs and/or Rb,
0.2≤a≤1,
0.5≤b≤10,
0≤c≤10,
0≤d≤10,
2≤c+d≤10
0≤e≤2,
0≤f≤10,
0≤g≤0.5
y=a number determined by the valence and abundance of elements other than oxygen in (la) required to maintain electrical neutrality.

Preference is given to catalysts whose catalytically active oxide composition comprises only Co from among the two metals Co and Ni (d=0). X¹ is preferably Si and/or Mn and X² is preferably K, Na and/or Cs, with particular preference being given to X² being K. Particular preference is given to a largely Cr(VI)-free catalyst.

The reaction temperature of the oxydehydrogenation is generally controlled by means of a heat transfer medium which is present around the reaction tubes. Possible liquid heat transfer media of this type are, for example, melts of salts or salt mixtures such as potassium nitrate, potassium nitrite, sodium nitrite and/or sodium nitrate and also melt of metals such as sodium, mercury and alloys of various metals. However, ionic liquids or heat transfer oils can also be used. The temperature of the heat transfer medium is in the range from 220 to 490° C. and preferably from 300 to 450° C. and particularly preferably from 330 to 420° C.

Owing to the exothermic nature of the reactions which proceed, the temperature in particular sections of the interior of the reactor during the reaction can be higher than the temperature of the heat transfer medium and a hot spot is thus formed. The position and height of the hot spot is determined by the reaction conditions, but can also be regulated via the dilution ratio of the catalyst bed or the flow of mixed gas. The difference between hot spot temperature and the temperature of the heat transfer medium is generally in the range from 1 to 150° C., preferably from 10 to 100° C. and particularly preferably from 20 to 80° C. The temperature at the end of the catalyst bed is generally from 0 to 100° C. above, preferably from 0.1 to 50° C. above, particularly preferably from 1 to 25° C. above, the temperature of the heat transfer medium.

The oxydehydrogenation can be carried out in all fixed-bed reactors known from the prior art, for example in a tray oven, in a fixed-bed tube reactor or shell-and-tube reactor or in a plate heat exchanger reactor. A shell-and-tube reactor is preferred.

The oxidative dehydrogenation is preferably carried out in fixed-bed tube reactors or fixed-bed shell-and-tube reactors. The reaction tubes are (like the other elements of the shell-and-tube reactor) generally made of steel. The wall thickness of the reactor tubes is typically from 1 to 3 mm. Their internal diameter is generally (uniformly) from 10 to 50 mm or from 15 to 40 mm, frequently from 20 to 30 mm. The number of reaction tubes accommodated in the shell-and-tube reactor is generally at least 1000, or 3000, or 5000, preferably at least 10 000. The number of reaction tubes accommodated in the shell-and-tube reactor is frequently from 15 000 to 30 000 or up to 40 000 or up to 50 000. The length of the reaction tubes normally extends to a few meters, with a typical reaction tube length being in the range from 1 to 8 m, frequently from 2 to 7 m, often from 2.5 to 6 m.

Furthermore, the catalyst bed installed in the ODH reactor(s) R can consist of a single zone or of two or more zones. These zones can consist of pure catalyst or be diluted with a material which does not react with the feed gas or components of the product gas from the reaction. Furthermore, the catalyst zones can consist of all-active material and/or supported coated catalysts.

The product gas stream leaving the oxidative dehydrogenation generally comprises as yet unreacted 1-butene and 2-butene, oxygen and steam in addition to butadiene. As secondary components, it generally also comprises carbon monoxide, carbon dioxide, inert gases (mainly nitrogen), low-boiling hydrocarbons such as methane, ethane, ethene, propane and propene, butane and isobutane, possibly hydrogen and possibly oxygen-comprising hydrocarbons, known as oxygenates. Oxygenates can be, for example, formaldehyde, furan, acetic acid, maleic anhydride, formic acid, methacrolein, methacrylic acid, crotonaldehyde, crotonic acid, propionoic acid, acrylic acid, methyl vinyl ketone, styrene, benzaldehyde, benzoic acid, phthalic anhydride, fluorenone, anthraquinone and butyraldehyde.

The product gas stream at the reactor outlet is characterized by a temperature close to the temperature at the end of the catalyst bed. The product gas stream is then brought to a temperature of from 150 to 400° C., preferably from 160 to 300° C., particularly preferably from 170 to 250° C. It is possible to insulate the conduit through which the product gas stream flows or to use a heat exchanger in order to keep the temperature in the desired range. This heat transfer medium system can be any heat transfer medium system as long as the temperature of the product gas can be kept at the desired level by means of this system. As examples of a heat exchanger, mention may be made of spiral heat exchangers, plate heat exchangers, double-tube heat exchangers, multitube heat exchangers, boiler-spiral heat exchangers, boiler-jacketing exchangers, liquid-liquid contact heat exchangers, air heat exchangers, direct contact heat exchangers and finned tube heat exchangers. Since part of the high-boiling by-products comprised in the product gas can precipitate while the temperature of the product gas is adjusted to the desired temperature, the heat exchanger system should preferably have two or more heat exchangers. If two or more heat exchangers provided are arranged in parallel and divided cooling of the product gas obtained is thus possible in the heat exchangers, the amount of high-boiling by-products which precipitate in the heat exchangers decreases and the period of operation of these can therefore be increased. As an alternative to the abovementioned method, the two or more heat exchangers provided can be arranged in parallel. The product gas is fed to one or more but not all of the heat exchangers which are, after a particular period of operation, relieved by other heat exchangers. In this method, cooling can be continued and part of the heat of reaction can be recovered while, in parallel thereto, the high-boiling by-products precipitated in one of the heat exchangers can be removed. As an abovementioned coolant, it is possible to use a solvent which is able to dissolve the high-boiling by-products. Examples are aromatic hydrocarbon solvents such as toluene and xylenes, diethylbenzenes, triethylbenzenes, diisopropylbenzenes, triisopropylbenzenes. Particular preference is given to mesitylene. It is also possible to use aqueous solvents. These can be made either acidic or alkaline, for example an aqueous solution of sodium hydroxide.

A major part of the high-boiling secondary components and of the water is subsequently separated from the product gas stream by cooling and compression. Cooling is effected by contacting with a coolant. This stage win subsequently also be referred to as quench Q. This quench can consist of only one stage or of a plurality of stages. The product gas stream is thus brought directly into contact with a preferably organic cooling medium and cooled thereby. Suitable cooling media are aqueous coolants or organic solvents, preferably aromatic hydrocarbons, particularly preferably toluene, o-xylene, m-xylene, p-xylene or mesitylene, or mixtures thereof. All possible isomers of diethylbenzene, triethylbenzene, diisopropylbenzene and triisopropylbenzene and mixtures therefore can also be used.

Preference is given to a two-stage quench, i.e. stage Ca) comprises two cooling stages Ca1) and Ca2) in which the product gas stream b is brought into contact with the organic solvent.

In a preferred embodiment of the invention, the cooling stage Ca) is thus carried out in two stages, with the solvent loaded with secondary components from the second stage Ca2) being fed into the first stage Ca1). The solvent taken off from the second stage Ca2) contains a smaller amount of secondary components than the solvent taken off from the first stage Ca1).

A gas stream comprising n-butane, 1-butene, 2-butenes, butadiene, possibly oxygen, hydrogen, steam, small amounts of methane, ethane, ethene, propane and propene, isobutane, carbon oxides, inert gases and parts of the solvent used in the quench is obtained. Furthermore, traces of high-boiling components which are not separated off quantitatively in the quench can remain in this gas stream.

The product gas stream from the solvent quench is compressed in at least one compression stage K and subsequently cooled further in the cooling apparatus, forming at least one condensate stream. A gas stream comprising butadiene, 1-butene, 2-butenes, oxygen, steam, possibly low-boiling hydrocarbons such as methane, ethane, ethene, propane and propane, butane and isobutane, possibly carbon oxides and possibly inert gases remains. Furthermore, this product gas stream can also comprise traces of high-boiling components.

The compression and cooling of the gas stream can be carried out in one or more stages (n-stage). In general, the gas stream is compressed overall from a pressure in the range from 1.0 to 4.0 bar (absolute) to a pressure in the range from 3.5 to 20 bar (absolute). Each compression stage is followed by a cooling stage in which the gas stream is cooled to a temperature in the range from 15 to 60° C. The condensate stream can thus also comprise a plurality of streams in the case of multistage compression. The condensate stream consists mostly of water and possibly the organic solvent used in the quench. Both streams (aqueous and organic phase) can additionally comprise small amounts of secondary components such as low boilers, C₄-hydrocarbons, oxygenates and carbon oxides.

The gas stream comprising butadiene, n-butenes, oxygen, low-boiling hydrocarbons (methane, ethane, ethene, propane, propene, n-butane, isobutane), possibly steam, possibly carbon oxides and possibly inert gases and possibly traces of secondary components is fed as feed stream to the further treatment.

In a step D), incondensable and low-boiling gas constituents comprising oxygen, low-boiling hydrocarbons (methane, ethane, ethene, propane, propene), carbon oxides and inert gases are separated as gas stream from the process gas stream in an absorption column A by absorption of the C₄-hydrocarbons in a high-boiling absorption medium and subsequent desorption of the C₄-hydrocarbons. Step D) preferably comprises the steps Da1), Da2) and Db):
Da1) absorption of the C₄-hydrocarbons comprising butadiene and n-butenes in a high-boiling absorption medium, giving an absorption medium stream loaded with C₄-hydrocarbons and the gas stream,
Da2) removal of oxygen from the absorption medium loaded with C₄-hydrocarbons from step Da) by stripping with an incondensable gas stream, giving an absorption medium stream loaded with C₄-hydrocarbons, and
Db) desorption of the C₄-hydrocarbons from the loaded absorption medium stream, giving a C₄ product gas stream consisting essentially of C₄-hydrocarbons.

For this purpose, the gas stream is brought into contact with an inert absorption medium in the absorption stage Da1) and the C₄-hydrocarbons are absorbed in the inert absorption medium, giving an absorption medium loaded with C₄-hydrocarbons and an offgas comprising the remaining gas constituents. In a desorption stage, the C₄-hydrocarbons are liberated again from the high-boiling absorption medium.

The absorption stage can be carried out in any suitable absorption column known to those skilled in the art. The absorption can be carried out by simply passing the product gas stream through the absorption medium. However, it can also be carried out in columns or in rotational absorbers. These can operate in cocurrent, countercurrent or cross-current. The absorption is preferably carried out in countercurrent. Suitable absorption columns are, for example, tray columns having bubble cap trays, centrifugal trays and/or sieve trays, columns having structured packing, e.g. sheet metal packing having a specific surface area of from 100 to 1000 m²/m³ e.g. Mellapak® 250 Y, and columns packed with random packing elements. However, trickle and spray towers, graphite block absorbers, surface absorbers such as thick film and thin film absorbers and also rotational columns, plate scrubbers, crossed-spray scrubbers and rotational scrubbers are also possible.

In one embodiment, the gas stream comprising butadiene, n-butenes and the low-boiling and incondensable gas constituents is fed into the lower region of an absorption column. The high-boiling absorption medium is introduced in the upper region of the absorption column.

Inert absorption medium used in the absorption stage are generally high-boiling nonpolar solvents in which the C₄-hydrocarbon mixture to be separated off has a significantly greater solubility than the remaining gas constituents to be separated off. Suitable absorption media are comparatively nonpolar organic solvents, for example aliphatic C-Cis-alkanes, or aromatic hydrocarbons such as the middle oil fractions from paraffin distillation, toluene or ethers having bulky groups, or mixtures of these solvents, with a polar solvent such as 1,2-dimethyl phthalate being able to be added to these. Further suitable absorption media are esters of benzoic acid and phthalic acid with straight-chain C₁-C₈-alkanois, and also heat transfer oils such as biphenyl and diphenyl ether, their chloro derivatives and also triarylalkenes. One suitable absorption medium is a mixture of biphenyl and diphenyl ether, preferably having the azeotropic composition, for example the commercially available Diphyl®. This solvent mixture frequently comprises dimethyl phthalate in an amount of from 0.1 to 25% by weight.

In a preferred embodiment of the absorption stage Da1), the same solvent as in the cooling stage Ca) is used.

Preferred absorption media are solvents which have a solvent capability for organic peroxides of at least 1000 ppm (mg of active oxygen/kg of solvent). Preference is given to aromatic hydrocarbons, particularly preferably toluene, o-xylene, p-xylene and mesitylene, or mixtures thereof. All possible isomers of diethylbenzene, triethylbenzene, diisopropylbenzene and triisopropylbenzene and mixtures thereof can also be used.

At the top of the absorption column, a gas stream d comprising essentially oxygen, low-boiling hydrocarbons (methane, ethane, ethene, propane, propene), the hydrocarbon solvent, possibly C₄-hydrocarbons (butanes, butenes, butadiene), possibly inert gases, possibly carbon oxides and possibly steam is taken off. This stream is at least partly fed as recycle gas stream d2 to the ODH reactor. This allows, for example, the feed stream to the ODH reactor to be set to the desired C₄-hydrocarbon content. In general, optionally after separating off a purge gas stream, at least 10% by volume, preferably at least 30% by volume, of the gas stream d are recirculated as recycle gas stream d2 into the oxidative dehydrogenation zone.

In general, the recycle stream amounts to from 10 to 70% by volume, preferably from 30 to 60% by volume, of the sum of all streams fed into the oxidative dehydrogenation B).

The purge gas stream can be subjected to thermal or catalytic after-combustion. In particular, it can be utilized thermally in a power station.

At the bottom of the absorption column, residues of oxygen dissolved in the absorption medium are discharged by flushing with a gas in a further column. The remaining proportion of oxygen should be so small that the stream which leaves the desorption column and comprises butane, butene and butadiene now comprises only a maximum of 100 ppm of oxygen.

The stripping-out of the oxygen in step Da2) can be carried out in any suitable column known to those skilled in the art. Stripping can be carried out by simply passing incondensable gases, preferably gases which are not absorbed or only slightly absorbed in the absorption medium stream, e.g. methane, through the loaded absorption solution. C₄-hydrocarbons which are concomitantly stripped out are scrubbed back into the absorption solution in the upper part of the column by the gas stream being conveyed back into this absorption column. This can be achieved both by piping of the stripper column and by direct installation of the stripper column below the absorber column. Since the pressure in the stripper column section and the absorption column section is the same, this direct coupling can be employed. Suitable stripping columns are, for example, tray columns having bubble cap trays, centrifugal trays and/or sieve trays, columns have structured packing, e.g. sheet metal packing having a specific surface area of from 100 to 1000 m²/m³, e.g. Mellapak® 250 Y, and columns packed with random packing elements. However, trickle and spray towers and also rotational columns, plate scrubbers, crossed-spray scrubbers and rotational scrubbers are also possible. Suitable gases are, for example, nitrogen or methane.

In one embodiment of the process, stripping in step Da2) is carried out using a methane-comprising gas stream. In particular, this gas stream (stripping gas) comprises >90% by volume of methane.

The absorption medium stream loaded with C₄-hydrocarbons can be heated in a heat exchanger and subsequently fed into a desorption column. In one process variant, the desorption step Db) is carried out by depressurization and stripping of the loaded absorption medium by means of a stream of steam.

The absorption medium which has been regenerated in the desorption stage can be cooled in a heat exchanger. The cooled stream comprises water in addition to the absorption medium and this water is separated off in the phase separator.

The C₄ product gas stream consisting essentially of n-butane, n-butene, and butadiene generally comprises from 20 to 80% by volume of butadiene, from 0 to 80% by volume of n-butane, from 0 to 10% by volume of 1-butene, from 0 to 50% by volume of 2-butenes and from 0 to 10% by volume of methane, with the total amount adding up to 100% by volume. Furthermore, this stream can comprise small amounts of isobutane.

Part of the condensed overhead product comprising mainly C₄-hydrocarbons from the desorption column can be recirculated into the top of the column in order to increase the separation performance of the column.

The liquid or gaseous C₄ product streams leaving the condenser can subsequently be separated by extractive distillation in step E) using a solvent which is selective for butadiene into a stream comprising butadiene and the selective solvent and a stream comprising butanes and n-butenes.

In a preferred embodiment of the process of the invention, feeding of a reaction gas mixture whose composition is explosive into the oxidative dehydrogenation reactor is additionally prevented by means of a shutdown mechanism, with the shutdown mechanism being configured as follows:

a) an explosion diagram characteristic of the reaction gas mixture and in which explosive and nonexplosive compositions are delinearated from one another as a function of the composition of the reaction gas mixture is stored in a computer;

b) a data set is determined by determination of the amount and optionally composition of the gas streams fed into the reactor to produce the reaction gas mixture and this data set is fed into the computer;

c) the computer calculates an instantaneous operating point of the reaction gas mixture in the explosive diagram from the data set obtained in b);

d) if the distance of the operating point from the closest explosive limit is below a prescribed minimum value, the introduction of gas streams into the reactor is automatically interrupted.

The minimum value is preferably calculated from a statistical error analysis of the measured parameters necessary for calculating the operating point.

The process allows heterogeneously catalyzed gas-phase partial oxidations and oxidative dehydrogenations of at least one organic compound to be carried out with increased safety at oxygen contents of the reaction gas mixture which are ≥0.5 or r≥0.75, or ≥1, or ≥2, or ≥3, or ≥5, or ≥10, percentage points by volume above the limiting oxygen concentration. Here, the limiting oxygen concentration (LOC) is, as described above, the percentage by volume of molecular oxygen in the reaction gas mixture below which a combustion (explosion) initiated by a local ignition source (e.g. local overheating or spark production in the reactor) can no longer spread from the ignition source in the reaction gas mixture at a given pressure and temperature of said mixture, regardless of the quantity of the proportion by volume of the other constituents of the reaction gas mixture, namely, in particular, the organic compound to be oxidized and the inert diluent gas.

For safety reasons, it can be advantageous to store, as explosion diagram, not the course of the experimentally determined explosion limit but instead a switching curve which is shifted relative thereto by a safety margin in the computer. The safety margin is advantageously selected so that all error sources and measurement inaccuracies associated with determination of the operating point of the reaction gas mixture are taken into account. The safety margin can be determined both by an absolute error analysis or by a statistical error analysis. In general, a safety margin of from 0.1 to 0.4% points by volume of O₂ is sufficient.

Since the explosion behavior of butane and n-butenes is comparable and steam and nitrogen have a barely distinguishable effect on the explosion diagram of butane and/or butane, possible characteristic explosion diagrams to be recorded according to the invention in the computer are, for example:
a) the butenes/O₂-N₂ diagram;
b) the butanes/O₂-N₂ diagram;
c) the butenes/O₂-H₂O diagram;
d) the butanes/O₂-H₂O diagram;
e) the butenes/O₂-(N₂/H₂O) diagram;
f) the butanes/O₂—(Na/H₂O) diagram;

According to the invention, the butenes/O₂—N₂ explosion diagram is preferably stored in the computer.

In the experimental determination of the explosion diagram, a temperature which is not too far from the temperature range in which the partial oxidation takes place should be selected as temperature.

To calculate an informative instantaneous operating point of the reaction gas mixture in the explosion diagram, experimental determination of, for example, the following measured parameters is sufficient:
a) the amount of air fed into the reactor per unit time in standard m³;
b) the amount of butene-comprising feed gas fed into the reactor per unit time in standard m³;
c) the amount of steam and/or recycle gas fed into the reactor per unit time in standard m³;
d) the O₂ content of the recycle gas.

The oxygen content and nitrogen content of the air are known, the amount of butene-comprising feed gas and the amount of steam which is optionally concomitantly used are obtained as direct measurement result and the cycle gas is, apart from its oxygen content, assumed to consist exclusively of nitrogen. Should the recycle gas still comprise combustible constituents, this does not have a disadvantageous effect on the question of safety since the presence of these in the explosion diagram would merely mean a shift to the right of the real operating point relative to the calculated operating point. Steam comprised in small amounts in the recycle gas or carbon oxides comprised can be treated as nitrogen as far as safety relevance is concerned.

The measurement of the amounts of the gas streams fed into the reactor can be carried out using any measuring instrument suitable for this purpose. Possible measuring instruments of this type are, for example, all flow measuring instruments such as throttle instruments (e.g. orifice plates or Venturi tubes), displacement flow meters, float, turbine, ultrasonic, swirling and mass flow instruments. Owing to the low pressure drop, Venturi tubes are preferred according to the invention. To take account of pressure and temperature, the measured volume flows can be converted into standard m³.

The determination of the oxygen content of the recycle gas can, for example, be carried out in-line as described in DE-A 10117678. However, it can in principle also be carried out on-line by taking a sample of the product gas mixture coming from the oxidative dehydrogenation before it enters the target product separation (work-up) and analyzing this sample on-line in such a way that the analysis is carried out in a period of time which is shorter than the residence time of the product gas mixture in the work-up. That is to say, the amount of gas supplied to the analytical instrument has to be made sufficiently large by means of an analysis gas bypass and the piping system to the analytical instrument has to be made correspondingly small. An O₂ determination can of course also be carried out on the reaction gas instead of the recycle gas analysis. It is naturally also possible to carry out both. It is technically advantageous for the determination of the operating point for use in the inventive, safety oriented, memory-program control system (SSPS) to have a multichannel, preferably at least three-channel, configuration.

That is to say, each quantity measurement is preferably carried out by means of at least three fluid flow indicators (FFI) connected in series or in parallel. The same applies to the O₂ analysis. If one of the three operating points of the reaction gas mixture in the explosion diagram calculated from the three data sets goes below the prescribed minimum margin, the gas flow is automatically closed off, e.g. in the order air, then, with a time delay, butene-comprising feed gas and finally, if present, steam and/or recycle gas.

From the point of view of later restarting, it can be advantageous to continue to circulate steam and/or recycle gas.

As an alternative, an average operating point in the explosion diagram can also be calculated from the three individual measurements. If the distance of this from the explosion limit goes below a minimum value, an automatic shutdown is carried out as described above.

In principle, the method according to the invention can be employed not only for steady-state operation but also for the start-up and running down of the partial oxidation.

### EXAMPLES

The tube reactor (R) consists of stainless steel 1.4571, has an internal diameter of 29.7 mm and a length of 5 m and is filled with a mixed oxide catalyst (2500 ml). A thermocouple sheath (external diameter 6 mm) with thermocouples inside is installed in the center of the tube in order to measure the temperature profile in the bed. A salt melt flows around the tube in order to keep the outer wall temperature constant. A stream of butenes and butanes (a1), steam, air and oxygen-comprising recycle gas is fed to the reactor. Furthermore, nitrogen can be fed to the reactor.

The offgas (b) is cooled to 45° C. in a quenching apparatus (Q), with the high-boiling by-products being separated off. The stream is compressed to 10 bar in a compressor stage (K) and cooled to 45° C. again. A condensate stream c1 is discharged in the cooler. The gas stream c2 is fed to an absorption column (A). The absorption column is operated using mesitylene. A liquid stream enriched in organic products is taken off from the absorption column and a gaseous stream d is obtained at the top of the absorption column. The total work-up is designed so that water and the organic components are completely separated off. Part of the stream d is conveyed as recycle gas d2 back into the reactor.

### Example 1

FIG. 1 schematically shows the experimental setup. The recycle gas stream d2 is set to 5025 standard l/h and kept constant. The oxygen concentration in the recycle gas is 7.5% by volume, and is therefore approximately the same as in later steady-state operation. 285 Standard l/h of steam are fed to the reactor. A stream a1 consisting of 51% by volume of butenes and 49% by volume of butanes is then fed to the reactor. Beginning with a flow of 250 standard l/h of butenes/butanes, the flow is increased stepwise over a period of 25 minutes. After 25 minutes, the butenes/butanes stream a1 is 420 standard l/h, and at the same time the recycle gas stream d2 is reduced and after 25 minutes is 4820 standard l/h. The volume flow of the stream a1 is then increased to 630 standard l/h and at the same time air is introduced at an initial flow rate of 1338 standard l/h, and at the same time the recycle gas stream d2 is reduced to 3270 standard l/h. After 35 minutes, the volume flow rate of the stream a1 is increased to 845 standard l/h in a further step and at the same time the volume flow of air is increased to 2695 standard l/h, and at the same time the recycle gas stream d2 is reduced to 1700 standard l/h. A final value of 12.5% by volume of oxygen is obtained at the reactor inlet, with the oxygen coming both from the recycle gas stream d2 and from the air fed in. The total gas flow is kept approximately constant during the entire start-up procedure by reducing the recycle gas stream d2. The height of the hot spots can readily be regulated and controlled by addition of the butenes/butanes stream a1.

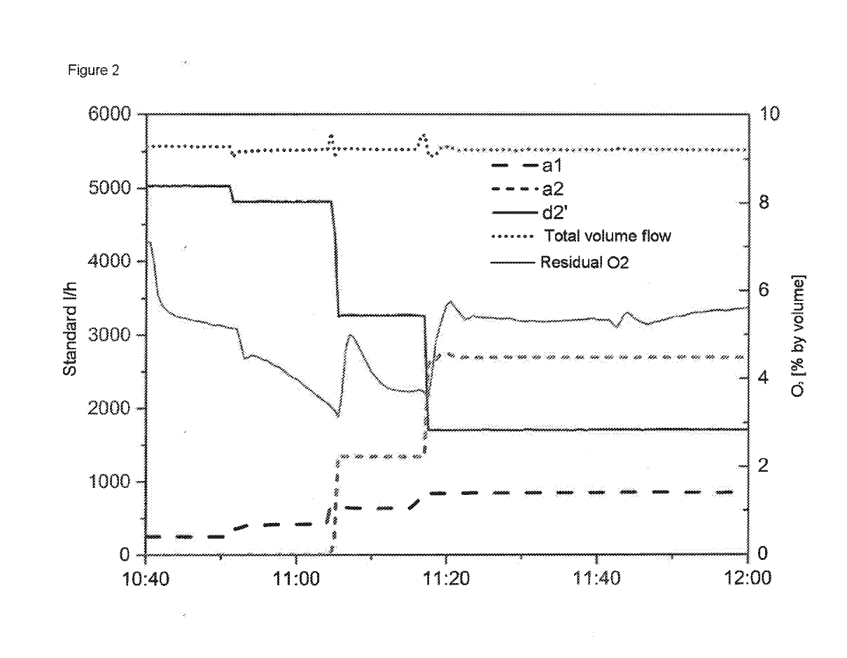

FIG. 2 shows the volume flow rates of C₄-hydrocarbons a1 and oxygen-comprising gas a2 and the resulting residual oxygen content at the outlet from the quench during the start-up procedure according to the invention. The 0₂ concentration in the feed is initially 7.5% by volume in order to simulate the 0₂ concentration in the recycle gas stream d2. Additional inert gas (N₂) for dilution is not required.

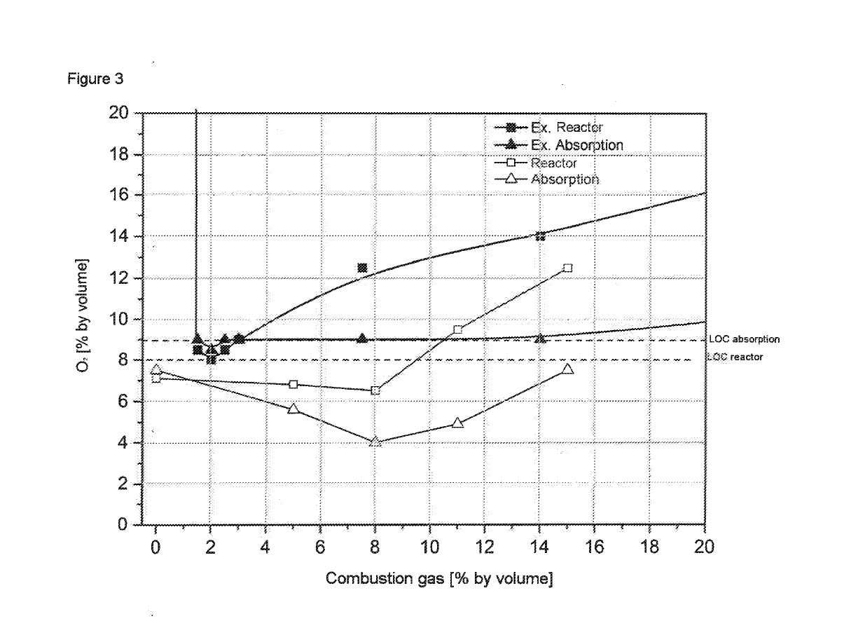

FIG. 3 shows the concentration curves for butanes/butenes (combustion gas), oxygen and the remaining gas components (100%−c_{combustion gas}−c_{O2}) upstream of the reactor (“reactor”) and also between quench and compression stage (“absorption”) together with the explosion diagrams for the reactor (“ex. reactor”) and the absorption column (“ex. absorption”). All concentrations are given in % by volume. The concentration of the combustion gas is plotted on the ordinate, and the concentration of oxygen is plotted on the abscissa. Immediately before the introduction of the butenes/butanes stream a1 is commenced, the oxygen concentration upstream and downstream of the reactor is, due to the dilution with steam, 7.1% by volume between quench and absorption column and 7.5% by volume in the recycle gas. Up to immediately before the introduction of air and while the combustion gas flow is being increased, the oxygen concentration between quench and absorption column decreases to about 4% by volume. After the introduction of air and while the combustion gas flow is being increased to the final value, the oxygen concentration upstream of the reactor rises to 12.5% by volume, downstream of the reactor to 6% by volume and between quench and absorption column to about 7.8% by volume, but without crossing over into the explosive range. Before air is fed in, the oxygen concentration upstream and downstream of the reactor and also between quench and absorption column cannot exceed the value in the recycle gas. A safe start-up can thus be ensured.

### Comparative Example 1

The reactor and the work-up section are firstly flashed with a stream of 1000 standard l/h of nitrogen. After one hour, the measured oxygen content downstream of the reactor and in the recycle gas is less than 0.5% by volume. 240 Standard l/h of air and 1000 standard l/h of nitrogen are then introduced into the reactor. The recycle gas stream is set to 2190 standard l/h. The recycle gas stream is kept constant. After 20 minutes, the oxygen concentration in the recycle gas stream is 4.1% by volume. The introduction of air and of nitrogen into the reactor are stopped simultaneously and 225 standard l/h of steam are fed into the reactor. Air and a stream consisting of 80% by volume of butenes and 20% by volume of butanes is then fed into the reactor, with the ratio of air stream to butenes/butanes stream being regulated so that this ratio remains constant at about 3.75. Beginning with a flow of 44 standard l/h of butenes/butanes and 165 standard l/h of air, these streams are increased at a constant ramp over a period of one hour and after one hour are 440 standard l/h of butenes/butanes and 1650 standard l/h of air. The recycle gas stream is kept constant during the entire start-up procedure and is 2190 standard l/h.

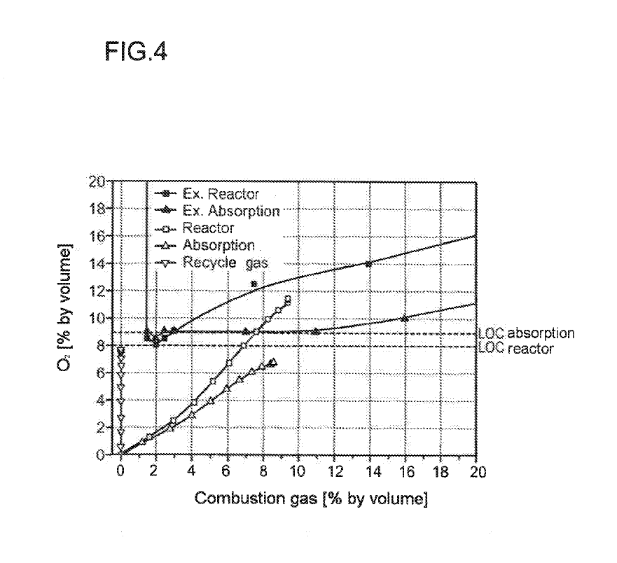

The plant is operated for 4 days, with a steady state in which the concentrations of the gas components change by not more than 5%/h being established. The concentration curve for butanes/butenes (combustion gas), oxygen and the remaining gas components (100%−c_{combustion gas}−c_{O2}) upstream of the reactor (“reactor”) and also between quench and compression stage (“absorption”) and in the recycle gas (“recycle gas”) is shown together with the explosion diagrams for the reactor (“ex. reactor”) and the absorption column (“ex. absorption”) in FIG. 4. All concentrations are given in % by volume. The concentration of the combustion gas is plotted on the ordinate, and the concentration of oxygen is plotted on the abscissa. Immediately before the introduction of combustion gas (butenes and butanes) is commenced, the oxygen concentration upstream and downstream of the reactor, between quench and absorption column and in the recycle gas is 4.1% by volume. While the combustion gas flow is being increased to the final value, the oxygen concentration in the recycle gas increases to a final value of about 7.5% by volume. The oxygen concentration also increases upstream of the reactor and between quench and absorption column, but without crossing over into the explosion range. A safe start-up can thus be ensured.

Comparative example 1 corresponds to example 1 of WO2015/104397. The explosion range is avoided in the reactor and the work-up. However, the procedure is less advantageous than the start-up procedure according to the invention since the oxygen content of the recycle gas available is reduced only by dilution with an inert gas and is subsequently increased again by introduction of an additional oxygen-comprising gas (air). However, the introduction of inert gas incurs additional costs, as does the introduction of an oxygen-comprising gas (air) since this has to be compressed to the required pressure.

### Comparative Example 2

The reactor is, as in comparative example 1, firstly flushed with a stream of 1000 standard l/h of nitrogen. After one hour, the measured oxygen content downstream of the reactor and in the recycle gas is less than 0.5% by volume. 620 Standard l/h of air and 1000 standard l/h of nitrogen are then introduced into the reactor. The recycle gas stream is set to 2190 standard l/h and kept constant. After 20 minutes, the oxygen concentration in the recycle gas is 7.9% by volume. The oxygen concentration in the recycle gas stream is thus approximately the same as in later steady-state operation, cf. table 1. Introduction of air and nitrogen into the reactor are stopped simultaneously. 225 Standard l/h of steam are fed into the reactor. Air and a stream consisting of 80% by volume of butenes and 20% by volume of butanes are then fed into the reactor, with the ratio of air stream to butenes/butanes stream being regulated in such a way that it is constant at about 3.75. Beginning with a flow of 44 standard l/h of butenes/butanes and 165 standard l/h of air, the streams are increased at a constant ramp over a period of one hour. After one hour, the butenes/butanes stream is 440 standard l/h and the air stream is 1650 standard l/h. The recycle gas stream is kept constant during the entire start-up procedure and is 2190 standard l/h.

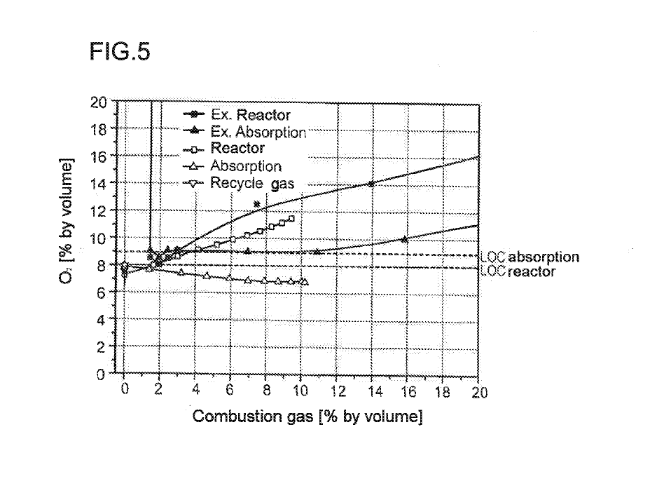

The concentration curve for butanes/butenes (combustion gas), oxygen and the remaining gas components (100%−c_{combustion gas}−c_{O2}) upstream of the reactor (“reactor”) and also between quench and compression stage (“absorption”) and in the recycle gas (“recycle gas”) is shown together with the explosion diagrams for the reactor (“ex. reactor”) and the absorption column (“ex. absorption”) in FIG. 5. All concentrations are given in % by volume. The concentration of the combustion gas is plotted on the ordinate, and the concentration of oxygen is plotted on the abscissa. Immediately before the introduction of combustion gas (butenes and butanes) is commenced, the oxygen concentration upstream and downstream of the reactor, between quench and absorption column and in the recycle gas is 7.9% by volume. While the combustion gas flow is being increased to the final value, the oxygen concentration in the recycle gas increases only slightly to a final value of about 7.6% by volume. The oxygen concentration increases upstream of the reactor and between quench and absorption column, and it can be seen that the distance from the explosion range in the reactor is very small during start-up of the reactor. Safe process operation is difficult to achieve here.

1. A process for preparing butadiene from n-butenes, which has a start-up phase and an operating phase and the operating phase of the process comprises the steps:
A) provision of a feed gas stream a1 comprising n-butenes;
B) introduction of the feed gas stream a1 comprising n-butenes, an oxygen-comprising gas stream a2 and an oxygen-comprising recycle gas stream d2 into at least one oxidative dehydrogenation zone and oxidative dehydrogenation of n-butenes to butadiene, giving a product gas stream b comprising butadiene, unreacted n-butenes, steam, oxygen, low-boiling hydrocarbons, high-boiling secondary components, possibly carbon oxides and possibly inert gases;
C) cooling and compression of the product gas stream b and condensation of at least part of the high-boiling secondary components, giving at least one aqueous condensate stream c1 and a gas stream c2 comprising butadiene, n-butenes, steam, oxygen, low-boiling hydrocarbons, possibly carbon oxides and possibly inert gases;
D) introduction of the gas stream c2 into an absorption zone and separation of incondensable and low-boiling gas constituents comprising oxygen, low-boiling hydrocarbons, possibly carbon oxides and possibly inert gases as gas stream d from the gas stream c2 by absorption of the C₄ hydrocarbons comprising butadiene and n-butenes in an absorption medium, giving an absorption medium stream loaded with C₄ hydrocarbons and the gas stream d, and recirculation, optionally after a purge gas stream p has been separated off, of the gas stream d as recycle gas stream d2 to the oxidative dehydrogenation zone,
where the start-up phase comprises the steps, in the order i) to iv):
i) introduction of a gas stream d2′ having a composition corresponding to the recycle gas stream d2 in the operating phase into the dehydrogenation zone and setting of the recycle gas stream d2 to at least 70% of the total volume flow in the operating phase;
ii) optionally additional introduction of a steam stream a3 into the dehydrogenation zone;
iii) additional introduction of the feed gas stream a1 comprising butenes at a lower volume flow than in the operating phase and raising of this volume flow until at least 50% of the volume flow of the feed gas stream a1 in the operating phase has been attained, with the total gas flow through the dehydrogenation zone corresponding to not more than 120% of the total gas flow during the operating phase;
iv) additional introduction, when at least 50% of the volume flow of the feed gas stream a1 comprising butenes in the operating phase has been attained, of an oxygen-comprising stream a2 at a lower volume flow than in the operating phase and raising of the volume flows of the feed gas streams a1 and a2 until the volume flows in the operating phase have been attained, with the total gas flow through the dehydrogenation zone corresponding to not more than 120% of the total gas flow during the operating phase.

2. The process according to claim 1, wherein the recycle gas stream d2 is set to from 95 to 105% of the total volume flow in the operating phase.

3. The process according to claim 1, wherein, in step iii), the volume flow of the feed gas stream comprising butenes is increased to not more than 75% of the volume flow in the operating phase.

4. The process according to claim 1, wherein, in step iv), initially only the volume flow of the oxygen-comprising gas stream a2 is increased until a ratio of oxygen to hydrocarbons which corresponds to the ratio of oxygen to hydrocarbons in the operating phase is attained and the volume flows of both the streams a1 and a2 are subsequently increased until 100% of the volume flow of the gas streams a1 and a2 in the operating phase is attained in each case.

5. The process according to claim 1, wherein the ratio of oxygen to hydrocarbons in the operating phase at an n-butenes content of from 50 to 100% by volume in the feed gas stream a1 is from 0.65:1 to 1.5:1.

6. The process according to claim 1, wherein the total gas flow comprising the streams a1, a2, d2 and optionally a3 through the dehydrogenation zone remains essentially constant during the steps (ii), (iii) and (iv) and corresponds to from 90 to 110% by volume of the total gas flow through the dehydrogenation zone during the operating phase.

7. The process according to claim 1, wherein the amount of steam in the dehydrogenation zone during the steps ii), iii) and iv) is from 0.5 to 10% by volume.

8. The process according to claim 1, wherein part of the recycle gas stream from one or more reactors operated in parallel for preparing butadiene from n-butenes by oxidative dehydrogenation, which are in the operating phase, is taken off as gas stream d2′.

9. The process according to claim 1, wherein the pressure in the dehydrogenation zone during the start-up phase is from 1 to 5 bar.

10. The process according to claim 1, wherein the pressure in the absorption zone during the start-up phase is from 2 to 20 bar.

11. The process according to claim 1, wherein step D) comprises the steps Da) and Db):
Da) separation of incondensable and low-boiling gas constituents comprising oxygen, low-boiling hydrocarbons, possibly carbon oxides and possibly inert gases as gas stream d from the gas stream c2 by absorption of the C₄-hydrocarbons comprising butadiene and n-butenes in an absorption medium, giving an absorption medium stream loaded with C₄-hydrocarbons and the gas stream d, and
Db) subsequent desorption of the C₄-hydrocarbons from the loaded absorption medium stream, giving a C₄ product gas stream d1.

12. The process according to claim 1 having the additional steps:
E) fractionation of the C₄ product stream d1 by extractive distillation using a solvent which is selective for butadiene to give a stream e1 comprising butadiene and the selective solvent and a stream e2 comprising n-butenes;
F) distillation of the stream f2 comprising butadiene and the selective solvent to give a stream g1 consisting essentially of the selective solvent and a butadiene-comprising stream g2.

13. The process according to claim 1, wherein the absorption medium used in step D) is an aromatic hydrocarbon solvent.

14. The process according to claim 1, wherein feeding of a reaction gas mixture whose composition is explosive into the oxidative dehydrogenation reactor is prevented by means of a shutdown mechanism, with the shutdown mechanism being configured as follows:
a) an explosion diagram characteristic of the reaction gas mixture and in which explosive and nonexplosive compositions are delinearated from one another as a function of the composition of the reaction gas mixture is stored in a computer;
b) a data set is determined by determination of the amount and optionally composition of the gas streams fed into the dehydrogenation zone to produce the reaction gas mixture and this data set is fed into the computer;
c) the computer calculates an instantaneous operating point of the reaction gas mixture in the explosive diagram from the data set obtained in b);
d) if the distance of the operating point from the closest explosive limit is below a prescribed minimum value, the introduction of gas streams into the dehydrogenation zone is automatically interrupted.
